(19) United States
(12) Patent Application Publication
(10) Pub. No.: US 2012/0004307 A1
(43) Pub. Date: Jan. 5, 2012

(54) ORAL TREATMENT OF DIGITAL ISCHEMIC LESIONS

(75) Inventors: Michael Wade, Research Triangle Park, NC (US); Kristan D. Rollins, Research Triangle Park, NC (US)

(73) Assignee: United Therapeutics Corporation

(21) Appl. No.: 13/160,034

(22) Filed: Jun. 14, 2011

Related U.S. Application Data
(60) Provisional application No. 61/354,949, filed on Jun. 15, 2010.

(51) Int. Cl. A61K 31/192; A61P 17/00; A61P 25/00; A61P 9/10

(52) U.S. Cl. 514/569

(57) ABSTRACT

Provided are methods of orally treating ischemic diseases and conditions, such as digital ulcers, associated with or caused by the ischemic diseases.

### RELATED APPLICATIONS

The present application claims priority to U.S. provisional application No. 61/354,949 filed Jun. 15, 2010, which is incorporated herein by reference in its entirety.

### FIELD

The present application relates to therapeutic methods and, in particular, to oral therapeutic methods for treating ischemic diseases and conditions associated with such diseases, such as digital ischemic lesions.

### SUMMARY

In one embodiment, the present invention relates to a method of treating a digital ischemic lesion and/or ameliorating or reducing at least one symptom and/or a functional deficit associated with a digital ischemic lesion: comprising orally administering to a subject in need thereof a formulation comprising an effective amount of treprostinil or a pharmaceutically acceptable salt thereof.

In another embodiment, the present invention relates to a method of treating a disease or condition selected from Raynaud's disease, scleroderma, including systemic sclerosis, and a combination thereof, comprising orally administering to a subject in need thereof a formulation comprising an effective amount of treprostinil or a pharmaceutically acceptable salt thereof.

### DRAWINGS

The application file contains at least one drawing executed in color. Copies of this patent application publication with color drawing(s) will be provided by the Office upon request and payment of the necessary fee.

FIG. 1 shows perfusion as assessed by laser Doppler imaging (LDI) improved after medication administration.

FIG. 2 shows median perfusion and drug concentration over time.

FIG. 3 shows median skin temperature and drug concentration over time.

FIG. 4 shows perfusion versus concentration areas under the curve (AUCs).

FIG. 5 provides summary of DISTOL PK and pilot perfusion study.

FIG. 6 schematically explains DISTOL-1 study design.

FIG. 7 schematically illustrates qualifying ulcers.

FIG. 8 presents results for net ulcer burden change.

FIG. 9 presents results for net ulcer burden outcome for ACA negative status (mean).

FIG. 10 presents results for net ulcer burden (mean within group).

FIG. 11 presents results for total ulcers.

FIG. 12 presents results for net ulcer burden at week 20 sorted by dose.

FIG. 13 presents VAS global impression of digital ulcers for total patient population.

FIG. 14 presents VAS global impression of digital ulcers for ACA negative patient subgroup.

FIG. 15 presents VAS for Ulcer related pain for total patient population.

FIG. 16 presents VAS for Ulcer related pain for ACA negative patient subgroup.

FIG. 17 presents physician VAS global impression of digital ulcers for total patient population.

FIG. 18 presents physician VAS global impression of digital ulcers for ACA negative patient subgroup.

FIG. 19 presents SHAQ's change at Week 20.

### DETAILED DESCRIPTION

ACA stands for anti-centromere autoantibody.
ATA stands for anti-topoisomerase autoantibody.
AUC stands for area under the curve.
BID means twice (two times) a day.
CHFS stands for CHFS stands for Cochin Hand Function Scale.
CLI stands for critical limb ischemia.
DU stands for digital ulcer(s).
ERA stands for endothelin receptor antagonist.
MTD stands for maximum tolerated dose.
PAD stands for periphepheral arterial disease.
PAH stands for pulmonary arterial hypertension.
PDEI or PDI stands for a phosphodiesterase inhibitor.
SF-36 stands short form-36 (Quality of Life Instrument).
SF-MPQ stands for Short Form McGill Pain Questionnaire.
SHAQ stands for Scleroderma Health Assessment Questionnaire.
SHAQ VAS stands for Scleroderma Health Assessment Questionnaire visual analog scale.
SSc stands for scleroderma (systemic sclerosis).
VAS stands for visual analogue scale or score.
Unless otherwise specified, “a” or “an” means “one or more”.

An oral formulation comprising treprostinil or a pharmaceutically acceptable salt thereof preferably includes a diethanolamine salt of treprostinil (treprostinil diethanolamine). The formulation may be effective for treating an ischemic disease or condition, such as scleroderma, including systemic sclerosis, or Raynaud's Phenomenon. The oral formulation comprising treprostinil diethanolamine may be also effective for treating one or more digital ischemic lesions, such as a digital ulcer or a necrotic lesion, ameliorating or reducing at least one symptom and/or functional deficit associated with a digital ischemic lesion. The term “digital ischemic lesion” refers to a lesion on a digit, i.e. a toe or a finger, of a subject, such as a human being. In many embodiments, the digital ischemic lesion may be caused by or associated with an ischemic disease or condition, such as scleroderma, including systemic sclerosis, or Raynaud's Phenomenon. The symptom that may be ameliorated and/or reduced may be, for example, a pain associated with a digital ischemic ulcer and/or scleroderma. In some embodiments, administering the oral formulation comprising treprostinil diethanolamine may provide amelioration or reduction of one or more functional deficits associated with a digital ischemic lesion. For example, in some embodiments, the formulation may provide amelioration or reduction in a hand function deficit, i.e. provide an improvement in the hand function of the treated subject.

In some embodiments, the treated subject may have a particular profile or status with respect to one or more antibodies associated with scleroderma and/or systemic sclerosis. For example, the treated subject may be a subject with a negative status with respect to one or more antibodies associated with scleroderma and systemic sclerosis or a subject with a positive status with respect to one or more antibodies associated with scleroderma and/or systemic sclerosis. Examples of antibodies associated with scleroderma and/or systemic sclerosis include, but not limited to, anti-endothelial cell antibodies, antifibroblast antibodies, anti-matrix metalloproteinase antibodies, and antifibrillin-1 antibodies; antinuclear antibodies, such as antitopoisomerase-I antibodies, anticentromere antibodies and antihistone antibodies; antinucleolar antibodies, such as anti-polymyositis/scleroderma antibodies, anti-Th/To antibodies, anti-U3-small nucleolar ribonucleoprotein particle antibodies, anti-U1-small nuclear ribonucleoprotein particle antibodies, anti-RNA polymerase antibodies, and anti-B23 antibodies; antiphospholipid antibodies, antineutrophil cytoplasmic antibodies, and antimitochondrial antibodies, see e.g. Chung and Utz, Current Rheumatology Reports 2004, 6:156-163, which is incorporated herein by reference in its entirety.

In some embodiments, the treated subject may be a subject with a negative anti-centromere autoantibody (ACA) status. In such a case, administering the oral formulation comprising treprostinil diethanolamine may reduce a net burden in the subject associated with the digital ulcer.

In some embodiments, oral administration of a formulation comprising treprostinil diethanolamine may provide at least one of the following favorable effects: a) increase digital perfusion in the subject, to whom the formulation is administered; b) increase digital skin temperature in the subject. For example, in some embodiments, the oral administration of the treprostinil diethanolamine formulation may increase digital perfusion in a human being to at least 200 units or at least 250 units from a starting value of below 180 units or below 160 units before the administering. The oral administration of the treprostinil diethanolamine formulation may increase a digital skin temperature in a human being to at least 29° C. or at least 29.5° C. or at least 30° C. or at least 30.5° C. from a value of below 28° C. or below 27.5° C. or below 27° C. or below 26.5° C. prior to the administering.

In some embodiments, oral administration of the treprostinil diethanolamine formulation may allow sustaining in the subject for at least 4 hours or for at least 6 hours or for at least 8 hours or at least 10 hours or at least 12 hours at least one of the following favorable effects: a) increase level of digital perfusion to whom the formulation is administered; b) increased digital skin temperature. For example, in some embodiments, the oral administration of the treprostinil diethanolamine formulation may allow sustaining a digital perfusion in a human being at least 200 units or at least 250 units for at least 4 hours or for at least 6 hours or for at least 8 hours or at least 10 hours or at least 12 hours. In some embodiments, the oral administration of the treprostinil diethanolamine formulation may allow sustaining a digital skin temperature in a human being to at least 29° C. or at least 29.5° C. or at least 30° C. or at least 30.5° C. for at least 4 hours or for at least 6 hours or for at least 8 hours or at least 10 hours or at least 12 hours.

### Treprostinil

Treprostinil is a chemically stable analog of prostacyclin, and as such is a potent vasodilator and inhibitor of platelet aggregation. The sodium salt of treprostinil, (1R,2R,3aS,9aS)-[[2,3,3a,4,9,9a-Hexahydro-2-hydroxy-1-[(3S)-3-hydroxyoctyl]-1H-benz[f]inden-5-yl]oxy]acetic acid monosodium salt, is sold as a solution for injection as Remodulin® which has been approved by the Food and Drug Administration (FDA) for treatment of pulmonary hypertension.

Treprostinil was first described in U.S. Pat. No. 4,306,075. U.S. Pat. Nos. 6,765,117 and 6,809,223 disclose stereoselective process for treprostinil synthesis. US patent application publication no. 2009/0163738 discloses an alternative process for preparation treprostinil. U.S. Pat. Nos. 7,384,978, 7,417,070 and 7,544,713 disclose oral forms of treprostinil. U.S. patent application publication no. 2010-0282622 discloses solid formulations of treprostinil. US application no. 13/151,465 filed Jun. 2, 2011 discloses an alternative process for preparation treprostinil.

The oral formulation comprises the diethanolamine salt of treprostinil (treprostinil diethanolamine). The diethanolamine salt of treprostinil can be in an amorphous or a crystalline state. In the crystalline state, the diethanolamine salt of treprostinil can have two polymorphs, with two forms, A and B, which are disclosed in U.S. Pat. Nos. 7,384,978, 7,417,070 and 7,544,713. In some embodiments, form B of treprostinil diethanolamine as disclosed in U.S. Pat. Nos. 7,384,978, 7,417,070 and 7,544,713 may be preferred. In many embodiments, the oral formulation may be in a dosage form, such as a tablet or a capsule. In some embodiments, the oral formulation may be in a form of a liquid or a suspension.

In some embodiments, the oral formulation may be a sustained release oral formulation, such as a sustained release osmotic formulation. The sustained release formulation may release treprostinil diethanolamine for at least 4 fours or at least 6 hours or at least 8 hours or at least 10 hours or at least 12 hours. In some embodiments, the sustained release formulation may allow sustaining a therapeutically effective concentration of treprostinil in a blood of the subject at least 4 fours or at least 6 hours or at least 8 hours or at least 10 hours or at least 12 hours. Treprostinil diethanolamine sustained release formulations, such as sustained release tablets and sustained release capsules, and methods of their making are disclosed, for example, in U.S. Pat. Nos. 7,384,978, 7,417,070 and 7,544,713.

The oral formulation preferably contains an effective amount treprostinil diethanolamine, i.e. an amount that allows to achieve a desired therapeutic effect. Besides treprostinil diethanolamine, the oral formulation may contain an appropriate oral excipient or an oral pharmaceutically acceptable carrier. Examples of appropriate oral excipients include, but not limited to, maltodextrin, sodium lauryl sulfate, magnesium stearate and/or xylitol.

In some embodiments, the oral formulation may contain at least 0.1 mg, or at least 0.2 or at least 0.5 mg or at least 1.0 mg of treprostinil diethanolamine.

In some embodiments, the oral formulation may contain at least 2 mg of treprostinil diethanolamine. In many embodiments, the oral formulation may contain more than 2 mg of treprostinil diethanolamine, such as up 4 mg of treprostinil diethanolamine. The use of such higher dose formulations may allow limiting the number of administering events to 3 or less or 2 or less per day, while still achieving the desired therapeutic effect.

In some embodiments, a daily dose of treprostinil diethanolamine may be from 0.1 mg to 20 mg or from 0.25 to 16 mg per day or any dose in between. In some embodiments, treprostinil diethanolamine may be administered 4 times per day or 3 times per day or twice per day or once per day.

The present invention can be illustrated in more detail by the following example, however, it should be understood that the present invention is not limited thereto.

### Example 1

Raynaud's phenomenon (RP) and a small vessel obliterative vasculopathy in systemic sclerosis (SSc) frequently lead to ischemic digital ulcers (DU).

Prior studies have demonstrated a therapeutic effect of IV prostacyclin analogs for ischemic DU, but oral prostacyclin analogs have had limited success; the lack of sustained plasma concentrations, particularly with immediate release oral prostacyclin analogues, may have limited the ability of prostanoids to produce sustained benefits in these earlier studies. Treprostinil diethanolamine (UT-15C) is an innovative salt form of the prostacyclin analog treprostinil for oral delivery as a sustained-release (SR) osmotic tablet.

### Objective

During a phase I PK trial of oral treprostinil in SSc patients, the goal was to quantify changes in perfusion as assessed by laser Doppler imaging (LDI) and to determine whether improvements in perfusion correlated with drug concentration.

### Methods

10 SSc patients with recent or active DU participated in the study.

Dosing: Oral treprostinil titrated up to 4 mg twice daily (BID) as tolerated over 6-8 weeks. Pharmokinetics (PK) and laser Doppler imaging (LDI) assessments: performed when subjects achieved the 2 mg and 4 mg (or maximally tolerated dose) doses. Subjects who did not reach a dose of 2 mg BID during the study had assessments performed only at the end of study visit. 8 serial measures of digital perfusion and drug concentration were obtained over 12 hours at each of the 2 study visits.

Repeated measures analyses were performed using random effects model. Dependent variables were perfusion and skin temperature. The predictor of interest was log-transformed drug concentration. Covariates with p-values less than 0.15 were kept in the model. All data was used, across time points, visits, and hands.

Table 1 presents baseline characteristics of study participants.

TABLE 1

| Variables | Sample (N = 10) |
|---|---|
| Age (years), mean (SD) | 44.3 (8.1) |
| Female Gender, no. (%) | 10 (100) |
| Race, no. (%) | |
| White | 9 (90) |
| Black | 1 (10) |
| Scleroderma subtype, no. (%) | |
| Limited | 7 (70) |
| Diffuse | 3 (30) |
| Scleroderma disease duration | 12.2 (7.0) |
| (years), mean (SD) | |
| Medication use* | |
| Calcium channel blockers | 9 (90) |
| ACE-inhibitor or ARB | 2 (20) |
| Aspirin | 3 (30) |
| Coumadin | 1 (10) |
| Statin | 1 (10) |
| PDE III inhibitor (cilostazol) | 1 (10) |

*No patient was on a nitrate, prostacyclin analog, endothelin receptor antagonist or PDE 5 inhibitor at baseline.

### Results

Nine subjects tolerated the 2 mg BID dose by the first PK visit, and 6 subjects tolerated the 4 mg BID dose by the 2nd PK visit. Two subjects completed the study at 0.5 mg BID and 1 mg BID, respectively.

Adverse effects were transient and typical of prostacyclin therapy: headache, jaw pain, photosensitivity, fatigue, insomnia, myalgias, nausea, emesis, diarrhea, abdominal bloating, edema, flushing.

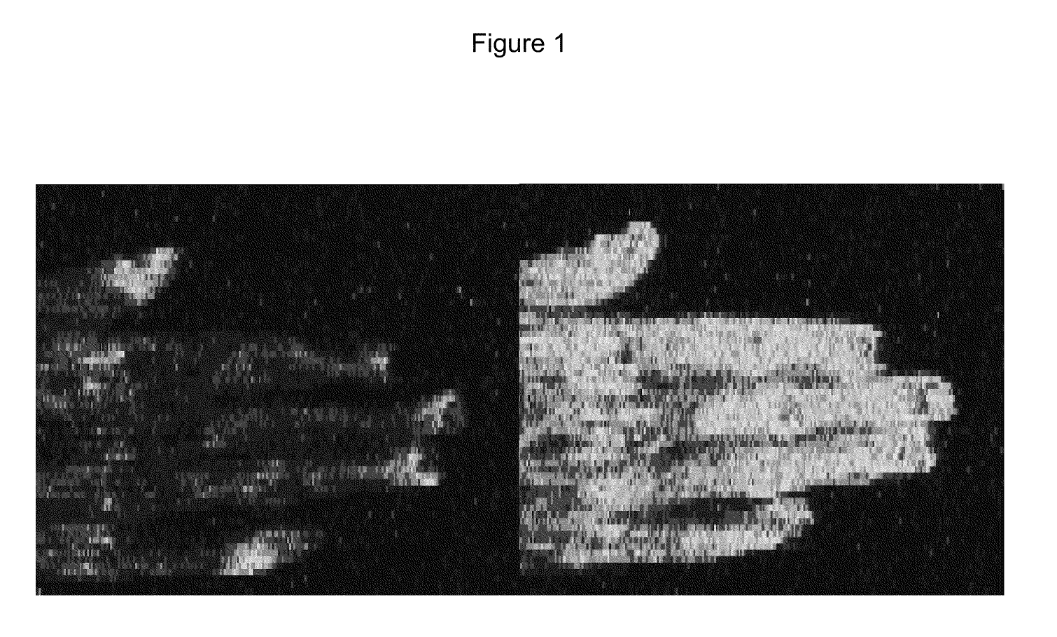

FIG. 1 shows perfusion as assessed by laser Doppler imaging (LDI) improved after medication administration. Sample images from Subject 6 at the 4 mg visit are shown. The baseline image at drug trough prior to administration of a 4 mg dose (left) corresponds to a mean perfusion of 89.3 units and a skin temperature of 25° C. Twelve hours after dosing, perfusion has improved to 298.3 units with a skin temperature of 32° C. (right).

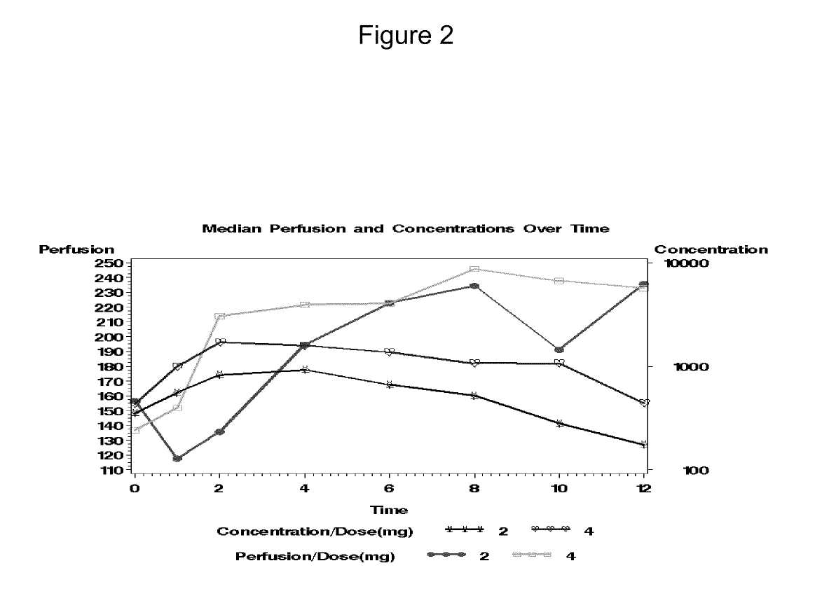

FIG. 2 shows median perfusion and drug concentration over time. Perfusion was positively associated with log-transformed plasma concentration at the 4 mg visit (but not the 2 mg visit), after adjusting for the individual time points of Doppler assessment at each PK visit (p=0.015).

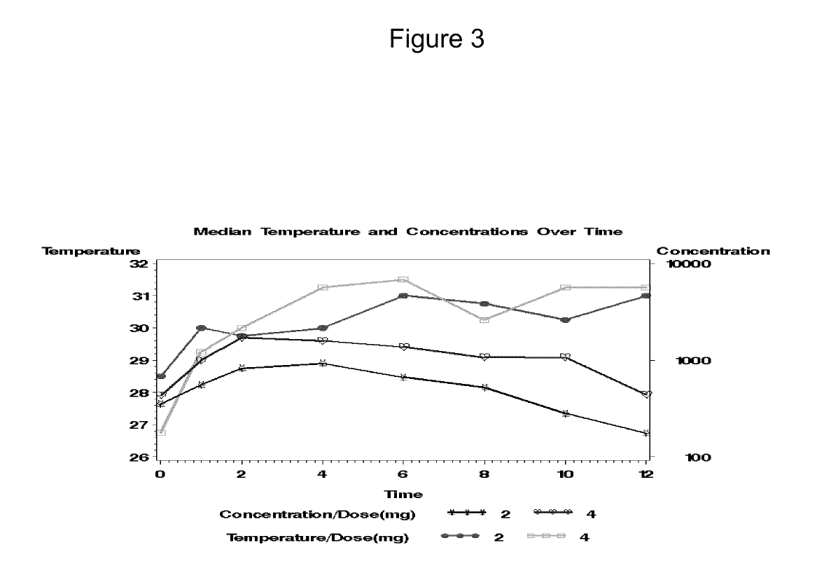

FIG. 3 shows median skin temperature and drug concentration over time. Digital skin temperature was positively associated with log-transformed plasma concentration at the 4 mg visit (but not the 2 mg visit), after adjusting for the individual time points of Doppler assessment at each PK visit (p=0.013).

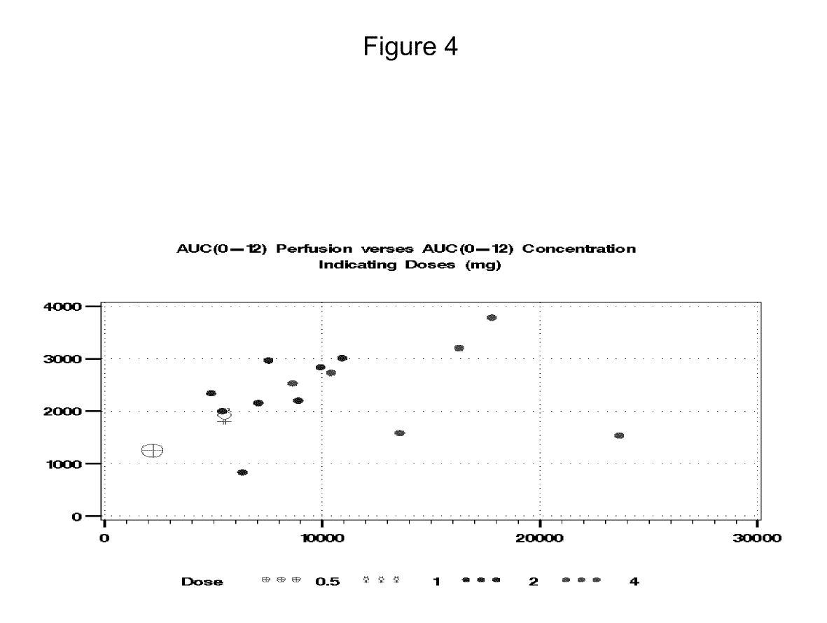

FIG. 4 shows perfusion versus concentration areas under the curve (AUCs). An increase in plasma concentration was observed with an increase in dose. An increase in perfusion was observed with an increase in drug exposure.

### SUMMARY

The oral sustained release formulation of treprostinil diethanolamine was absorbed to reach therapeutic levels and may provide new therapy for Raynaud's Phenomenon (RP) and the peripheral vascular disease of scleroderma.

Controlling for baseline perfusion, hour, and hand, concentration was a significant predictor of perfusion and temperature at the 4 mg visit. An increase in digital perfusion was observed with increased treprostinil blood concentrations, suggesting a dose-response relationship.

### Example 2

The following references may be useful for understanding the present invention:
1. Moehler E R, Vascular Medicine; 2000 5: 231-237
2. Berman S S, Vascular; 2006 14(3): 142-148
3. Chung L, Acad Dermatol; 2006 54(5): 880-882
4. Berman S S, (abstract and poster presentation) Soc. Clin. Vasc. Surgery; 2008
5. Korn J H, Arthritis & Rheumatism 2004; 50:3985-3893
6. Kowal-Bielecka O, Ann Rheum Dis; 2009 epub.

### Digital Ischemic Lesions in Scleroderma Treated with Oral Treprostinil Diethanolamine (DISTOL)

DISTOL program includes the following studies:
1) DISTOL PK study with pilot digital blood flow assessment (COMPLETED):
   (a) Open-label, two-part study
   (b) Cohort 1: Single 1 mg dose, Cohort 2: dose escalation up to 4 mg BID.
2) DISTOL-1 trial (COMPLETED):
   Randomized, Phase IIB study.
3) DISTOL-EXT Open-label extension trial (ongoing).

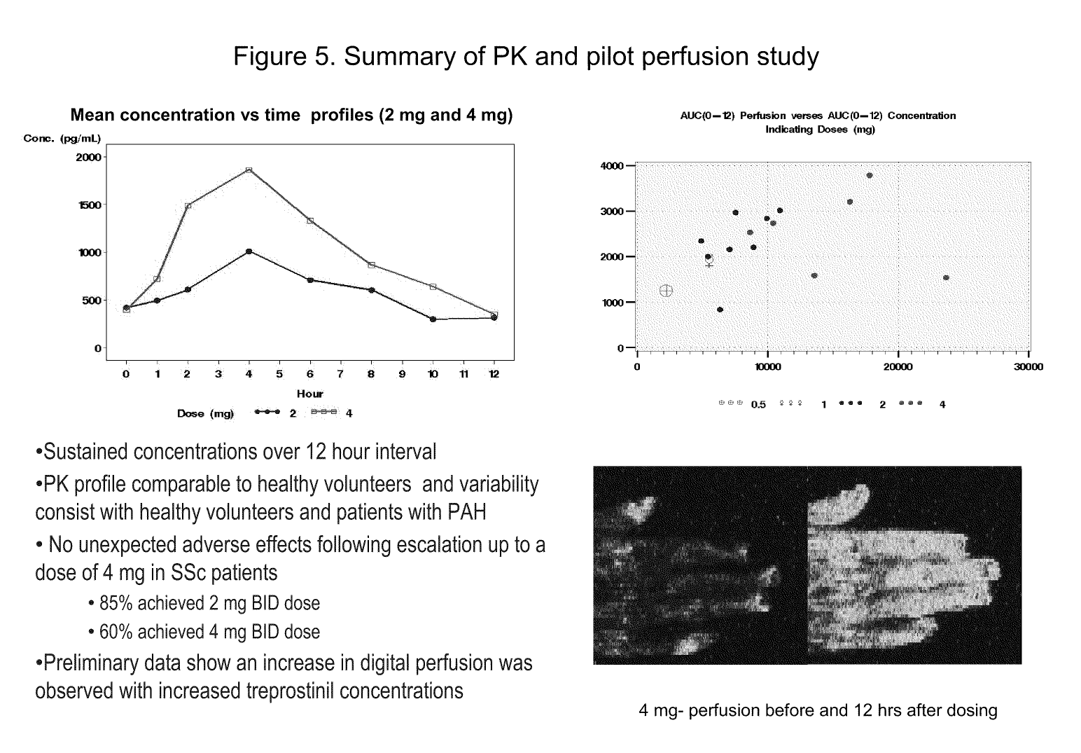

FIG. 5 provides summary of DISTOL PK and pilot perfusion study.

### DISTOL 1

### Study Objectives

Primary: To assess the effect of oral treprostinil in reducing net ulcer burden compared to placebo in patients with systemic sclerosis (SSc) as measured by the change in net ulcer burden from Baseline to Follow-up between treatment groups at 20 weeks.

Secondary: To assess the effect of treprostinil diethanolamine as compared to placebo on one or more of the following:
a) Time to complete healing of designated cardinal ulcer
b) Time to complete healing of all ulcers
c) Formation of new digital ulcers
d) Formation of new digital ulcers after week 5
e) Digital ulcer related pain (pain VAS and SF-MPQ)
f) Raynaud's phenomenon (SHAQ VAS)
g) Patient function (SHAQ)
h) Hand function (CHFS)
i) Quality of Life (SF-36)
j) Patient and physician global assessment (VAS)
k) Vascular and SSc associated biomarkers.

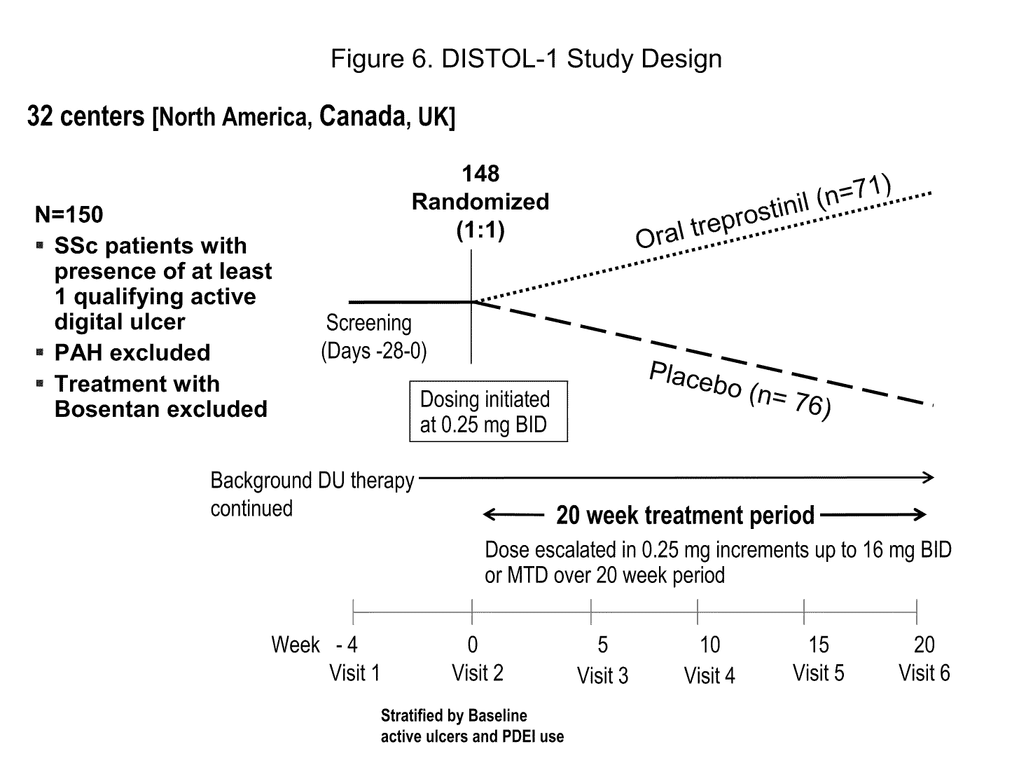

FIG. 6 schematically explains DISTOL-1 study design.

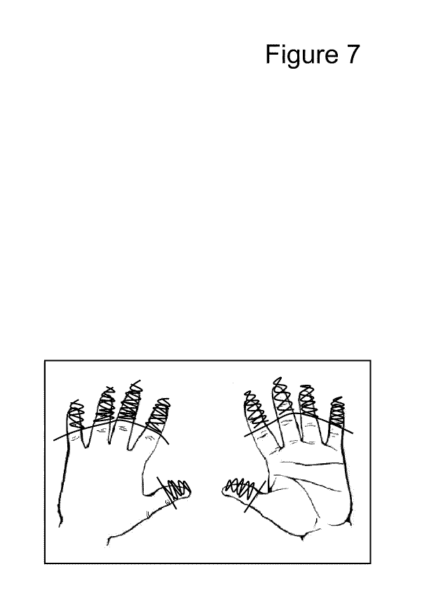

Ulcers qualifying for the study were defined as follows:
A denuded area with defined border and loss of epithelialization, loss of epidermis and dermis, which has the following properties:
   a) Vascular in origin;
   b) Without bone infection or calcinosis;
   c) Digital ulcer distal to the proximal interphalangeal joint;
   d) Volar to the median of the finger (palm side, see FIG. 7) (Does not include fissures, paronychia, extrusion of calcium, or ulcers over the metacarpophalangeal joints or elbows).

### Study Visits and Assessments

Baseline and Weeks 5, 10, 15, and 20:
Physician Digital Ulcer Assessment;
Physician Global Digital Ulcer VAS;
Scleroderma Health Assessment Questionnaire;
Cochin Hand Function Questionnaire;
McGill Pain Questionnaire;
SF-36;
Patient Digital Ulcer Pain VAS;
Patient Global Digital Ulcer VAS;
Patient Impression of Change Questionnaire;
Rodnan Skin Score (Baseline and Wk 20);
Physical Examination (Baseline and Wk 20);
Laboratory parameters (Baseline and Wk 20);
Serum Biomarker sample (Baseline and Wk 20).

### Statistical Considerations—Sample Size

Background rate in overall mean change of DUs in RAPIDS-2 trial was approximately −1.5 (sd=3) at 5 months. Allocation ratio of 1:1 between treprostinil diethanolamine and placebo, a fixed sample size of 128 patients would provide 80% power to detect a between-treatment difference at a significance level of 0.05 (two-sided hypothesis) assuming the active group leads to a change of −3 DUs. Target enrollment of 50 patients to complete 128 subjects. Estimated drop out rate 15%.

Primary analysis: effect of treprostinil versus placebo on change in net ulcer burden at Week 20 evaluated using non-parametric analysis of covariance within the framework of the extended Cochran-Mantel-Haenszel test.

Values for missing assessments imputed, see Table 2.

TABLE 2

| | Imputation Value Used: | |
|---|---|---|
| | Non- | |
| Event | parametric | Parametric |
| Death; progression of disease | Lowest rank | overall poorest |
| under study, addition of PDE | | relative change |
| inhibitor, ERA or prostacyclin | | |
| for 3 or more days | | |
| Premature discontinuation | Last observation | Last rank carried |
| (reason other than progression | carried forward | forward |
| of disease under study) or data | | |
| missing any other reason | | |

Planned Subgroup Analysis:
SSc classification (Diffuse/Limited)
Active Ulcers at Baseline (<2 active ulcers/>2 active ulcers)
Background PDEI use
Autoantibody Status

### Results

Table 3 presents demographics and baseline characteristics:

TABLE 3

| | Active | Placebo |
|---|---|---|
| | (n = 71) | (n = 76) |
| Age (mean, range) | 49.8 (19-82) | 47.8 (20-74) |
| Gender (M %/F %) | 24/76 | 28/72 |
| SSc Characteristics | | |
| Limited/Diffuse (%) | 56/44 | 72/28 |
| Years since scleroderma | 10.4 (0-35) | 10.7 (0-30) |
| diagnosis (mean, range) | | |
| Ulcer Characteristics | | |
| Active Ulcers at Baseline (mean, range) | 1.8 (1-6) | 1.6 (1-5) |
| Total Ulcers at Baseline (mean, range) | 2.7 (1-10) | 2.4 (1-7) |
| Autoantibody Status | | |
| Anti-Centromere Positive/Negative (%) | 27/56 | 38/45 |
| Anti-Topoisomerase Positive/ | 31/45 | 36/50 |
| Negative (%) | | |
| Unknown (%) | 10 | 11 |
| Background PDE Inhibitor Use | 12 (17%) | 13 (17%) |

Table 4 presents data for subject accountability.

TABLE 4

| | Active | Placebo |
|---|---|---|
| | (n = 72) | (n = 76) |
| Completed Study | 59 (82%) | 65 (86%) |
| Discontinuations - total (%) | 13 (18%) | 11 (14%) |
| Adverse Event | 9 (13%) | 6 (8%) |
| Consent Withdrawn | 0 | 1 (1%) |
| Protocol Violation | 2 (3%) | 2 (3%) |
| Lost to Follow-Up | 0 | 1 (1%) |
| Disease Progression | 1 (1%) | 1 (1%) |
| Never Dosed | 1 (1%) | 0 |

Table 5 presents study drug dosing—median (mg BID).

TABLE 5

| | |
|---|---|
| Active | Placebo |

| | | | | |
|---|---|---|---|---|
| Week 5 (n = 69/76) | 2.37 | (0.0-4.75) | 2.75 | (0.0-5.25) |
| Week 10 (n = 71/76) | 2.75 | (0.0-9) | 5.50 | (0.0-9) |
| Week 15 (n = 63/70) | 3 | (0.0-13.25) | 7.75 | (1-13.625) |
| Week 20 (n = 58/67) | 2.75 | (0.0-13) | 10 | (0.0-16.5) |

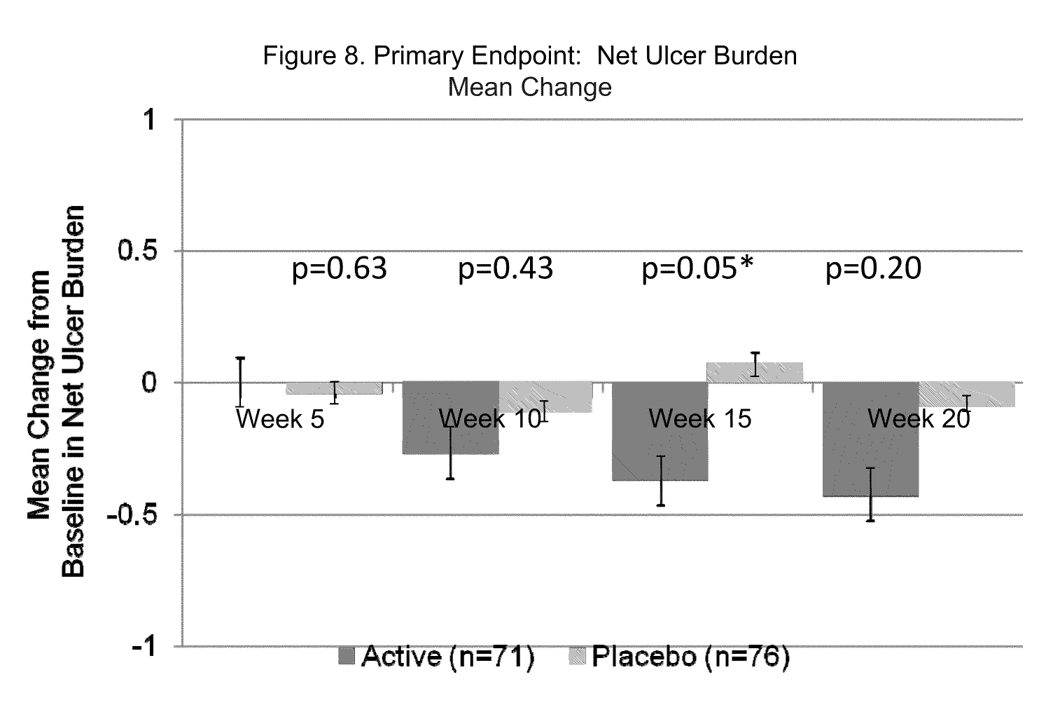

FIG. 8 presents results for net ulcer burden change.

Table 6 summarizes ulcer healing status.

TABLE 6

| | Active | Placebo |
|---|---|---|
| | (n = 71) | (n = 76) |
| Cardinal Ulcer Healed | 44 (62%) | 46 (61%) |
| Time to Cardinal Ulcer Healing | 76.3 +/− 35 | 83.2 +/− 37.9 |
| (mean days) | | |
| All Ulcers Healed | 35 (49%) | 31 (41%) |
| Time to all Ulcers Healed | 90.2 +/− 35.6 | 96.7 +/− 39.7 |
| (mean days) | | |

Table 7 presents results regarding formation new ulcers.

TABLE 7

| | Active | Placebo | |
|---|---|---|---|
| | (n =71) | (n = 76) | P value |
| Any new ulcers formed during study? | | | 0.59 |
| No new | 24 (34%) | 22 (29%) | |
| At least one new | 39 (55%) | 44 (58%) | |
| Any new ulcers formed after Week 5? | | | 0.40 |
| No new | 30 (42%) | 26 (34%) | |
| At least one new | 27 (38%) | 39 (51%) | |
| Mean # of new ulcers | 0.9 | 1.26 | |

Tables 8 and 9 provide results for net ulcer burden sorted by patients' subgroups.

TABLE 8

| | Mean Change in Net Ulcer |
|---|---|
| Subgroup | Burden Week 20 |
| ( N = Active/Placebo) | P value |
| Limited SSc (n = 42/55) | 0.42 |
| Diffuse SSc (n = 31/21) | 0.32 |
| ≦2 active ulcers at Baseline (n = 61/66) | 0.10 |
| >2 active ulcers at Baseline (n = 10/10) | 0.51 |
| PDEI background (n = 12/13) | 0.90 |
| No PDEI background (n = 59/63) | 0.21 |

TABLE 9

| | Mean Change in Net Ulcer |
|---|---|
| Subgroup | Burden Week 20 |
| (N = Active/Placebo) | P value |
| (ACA) Positive (n = 19/29) | 0.97 |
| (ACA) Negative (n = 40/34) | 0.01 |
| (ATA) Positive (n = 27/22) | 0.06 |
| (ATA) Negative (n = 32/38) | 0.58 |
| ACA Negative/ATA Positive (n = 20/15) | 0.013 |

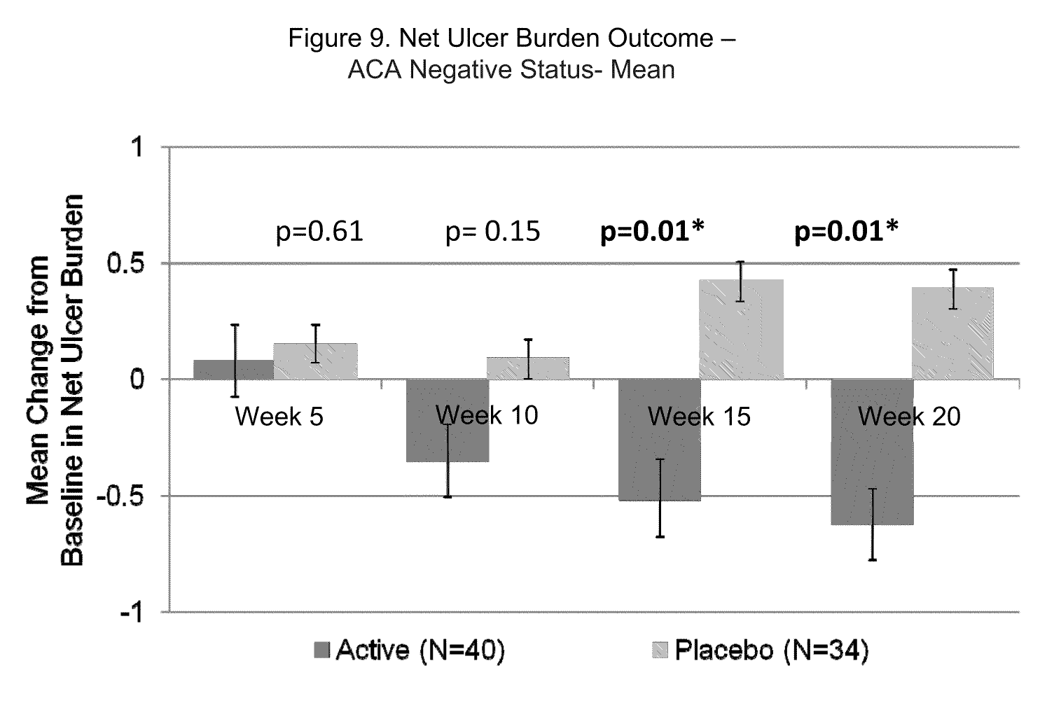

FIG. 9 presents results for net ulcer burden outcome for ACA negative status (mean).

Tables 10 and 11 present results for secondary endpoints.

TABLE 10

| | Change from Baseline |
|---|---|
| Endpoint | at Week 20 |
| (N = Active 71/placebo 76) | P value |
| Patient Global DU VAS* | 0.12 |
| Patient Digital Ulcer Pain VAS* | 0.31 |
| Physician Global DU VAS | 0.04 |
| Patient Impression of Change | |
| Digital Ulcers* | 0.21 |
| Raynaud's | 0.00004 |
| Overall | 0.019 |
| Cochin Hand Function | 0.47 |

TABLE 11

| | Change from Baseline |
|---|---|
| | at Week 20 |
| SHAQ Components | P value |
| Dressing/Grooming | 0.22 |
| Hygiene | 0.80 |
| Eating* | 0.15 |
| Grip | 0.05 |
| Activities | 0.37 |
| Hand Function | 0.05 |
| Aggregate* | 0.07 |
| DU VAS* | 0.13 |
| Pain VAS | 0.07 |
| Raynaud's VAS | 0.81 |
| Intestinal VAS | 0.06 |
| Breathing VAS | 0.01 |
| Overall VAS | 0.39 |

### Conclusion

In the primary analysis change, in net ulcer burden at Week 20 did not achieve statistical significance. However, statistically significant changes were observed in the ACA negative subset of patients.

The following secondary endpoints did achieve statistical significance: Physician Global VAS, SHAQ Grip, Hand Function and Breathing VAS components, Patient Impression of Change Overall and on Raynaud's. These data demonstrate that orally administered treprostinil diethanolamine provided symptomatic relief of pain (e.g. improvement in Raynaud's symptoms) and functional deficits associated with ischemic vascular disease in patients with scleroderma and digital ulcers. It can be expected that a compound that provides this level of functional and symptomatic improvement due to improvement in digital vascular pathology can also improve digital ulcer healing, further supporting the strong trend toward improvement in digital ulcer healing observed in the trial.

Improvement not reaching statistical significance was seen in Patient Global and Digital Ulcer pain VAS at Week 20.

### Additional Information

### DISTOL-1 Inclusion Criteria Summary

In order to be included in the study, each subject had to satisfy each of the following criteria:
Meet the American College of Rheumatology (ACR) criteria for systemic sclerosis (SSc);
be 18 years of age or older; and
have presence of at least one active digital ulcer at Baseline (meeting protocol defined qualifications).

### DISTOL-1 Exclusion Criteria Summary

Subjects satisfying at least one of the following criteria were excluded from the study:
Diagnosis of pulmonary arterial hypertension (PAH);
Diagnosis/simultaneously fulfills criteria for a second connective tissue disease
Use of systemic antibiotics within 2 weeks of Screening;
Weight less than 40 kg, intractable diarrhea, or severe malabsorption;
Blood pressure<90 mmHg systolic or <50 mmHg diastolic or history of postural hypotension or unexplained syncope;
Treatment with ERA treatment or statin (unless for hypercholesteremia);
Sympathectomy of the upper limb, involving the hand, performed within 12 months of Baseline (non-target limb or procedures not involving the hand within 6 months of Baseline);
Tobacco or nicotine use at any level within the past 6 months;
Use of prostacyclin within past 3 months.

### Background Therapy

Permitted background therapy included the following:
a) Calcium channel blocker (CCB), aspirin, alpha-1-antagonists, psychotropic vasodilator or angiotensin-converting enzyme (ACE), inhibitors, angiotensin receptor blockers (ARBs) and hemorrheologic agents;
b) Topical and systemic antibiotic use is permitted as medically warranted over the course of the study;
c) phosphodiesterase inhibitors, if stable for 6 months prior to entry.

Prohibited background therapy: ERA, statins (unless for hypercholestermia), oral or topical products containing nitric oxide, other prostanoids, Regranex.

### ADDITIONAL RESULTS

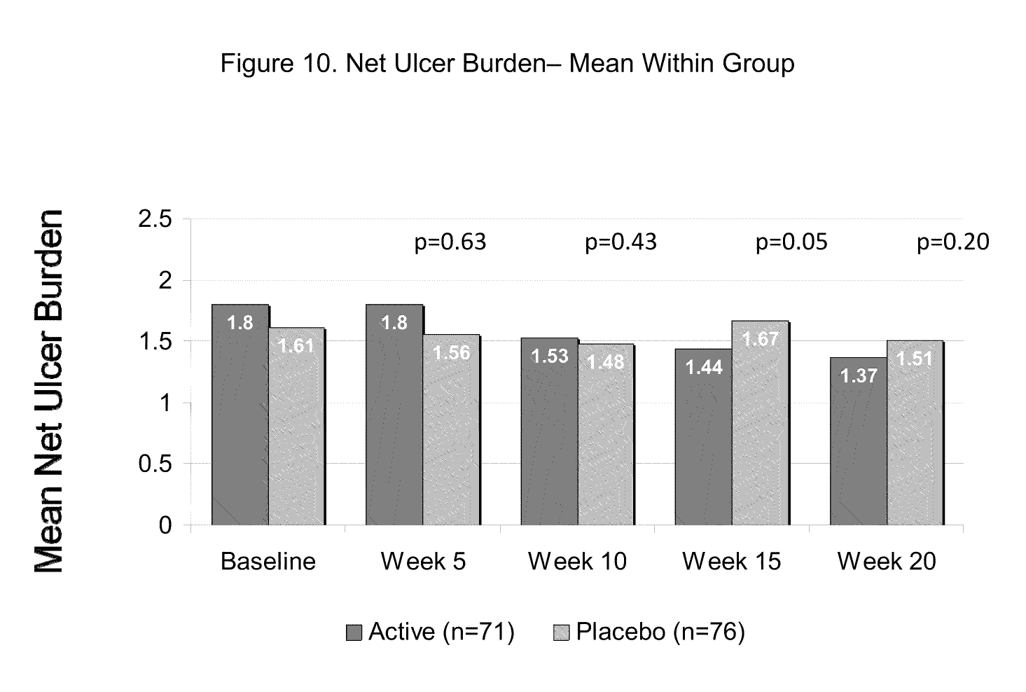

FIG. 10 presents results for net ulcer burden (mean within group).

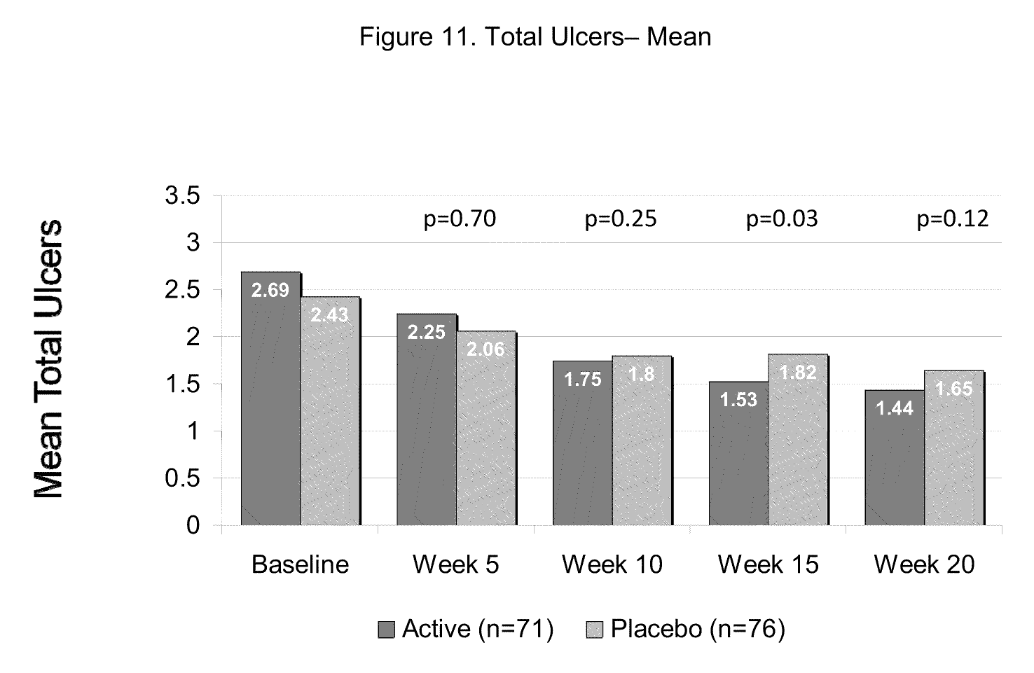

FIG. 11 presents results for total ulcers.

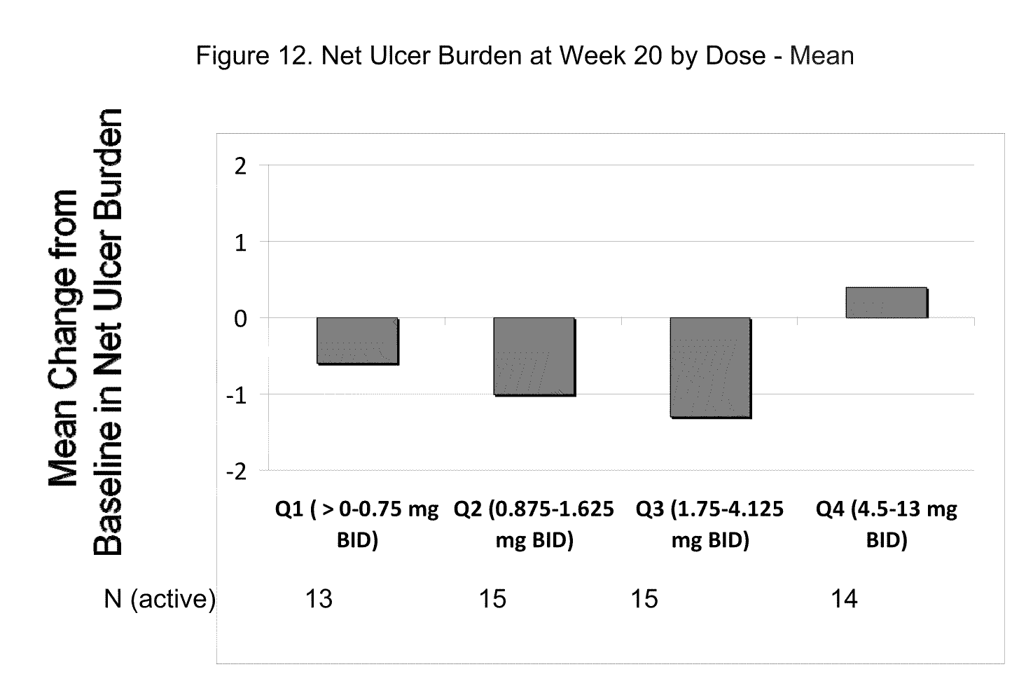

FIG. 12 presents results for net ulcer burden at week 20 sorted by dose.

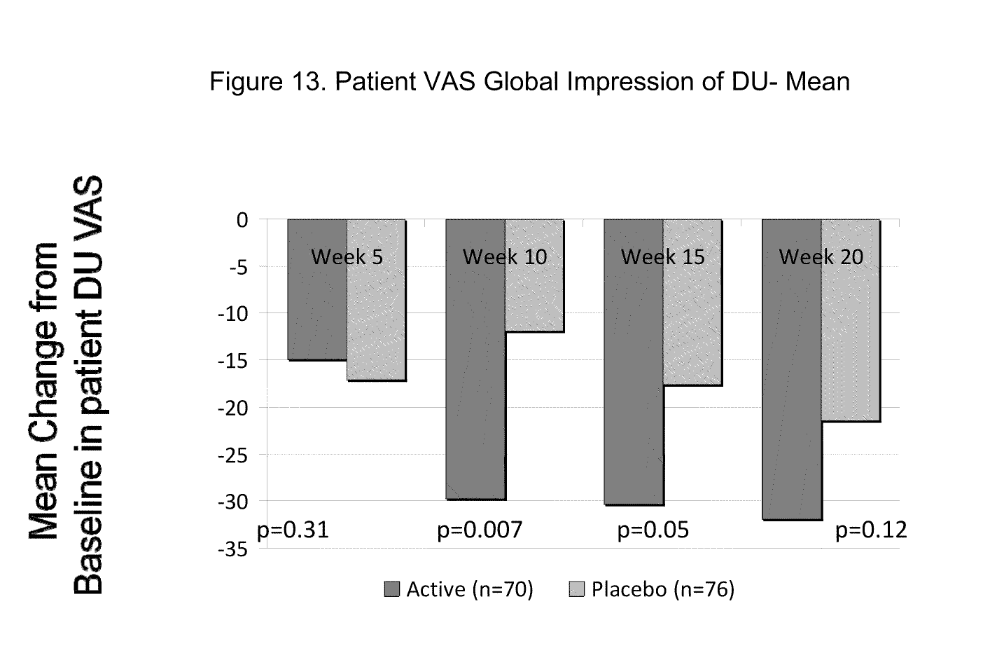

FIG. 13 presents VAS global impression of digital ulcers for total patient population.

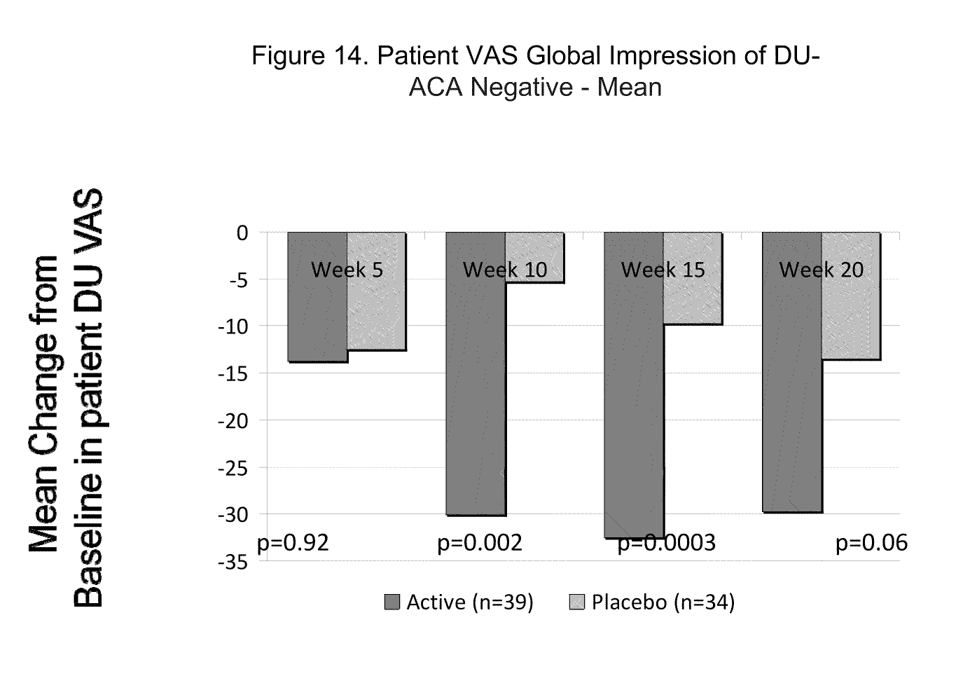

FIG. 14 presents VAS global impression of digital ulcers for ACA negative patient subgroup.

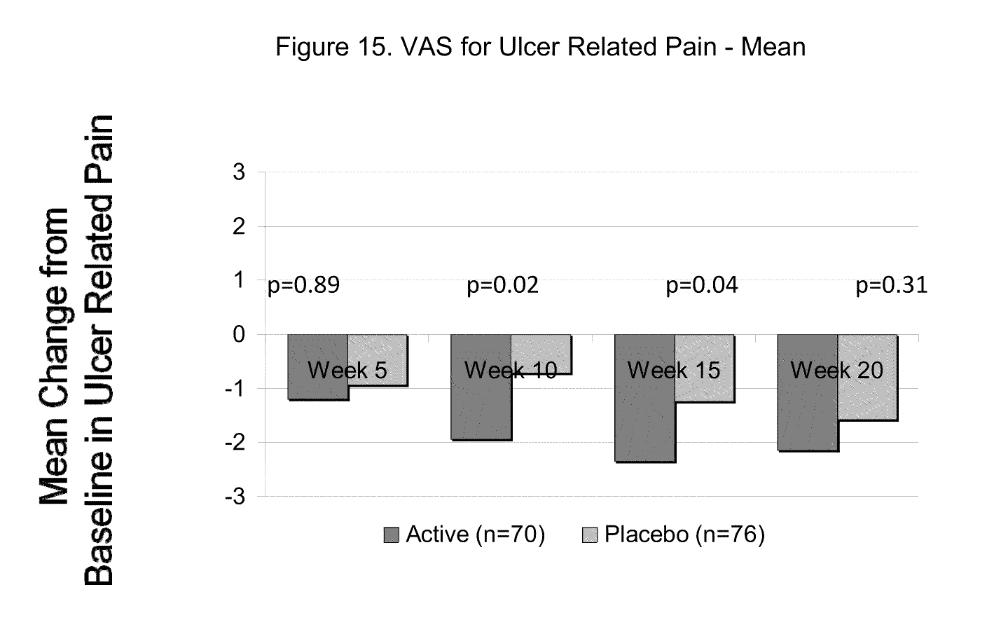

FIG. 15 presents VAS for Ulcer related pain for total patient population.

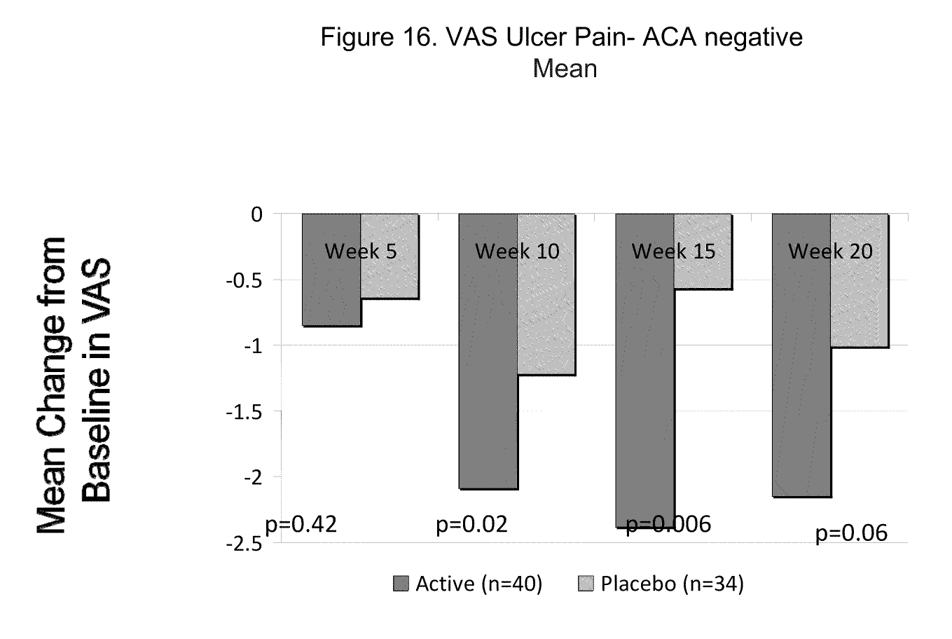

FIG. 16 presents VAS for Ulcer related pain for ACA negative patient subgroup.

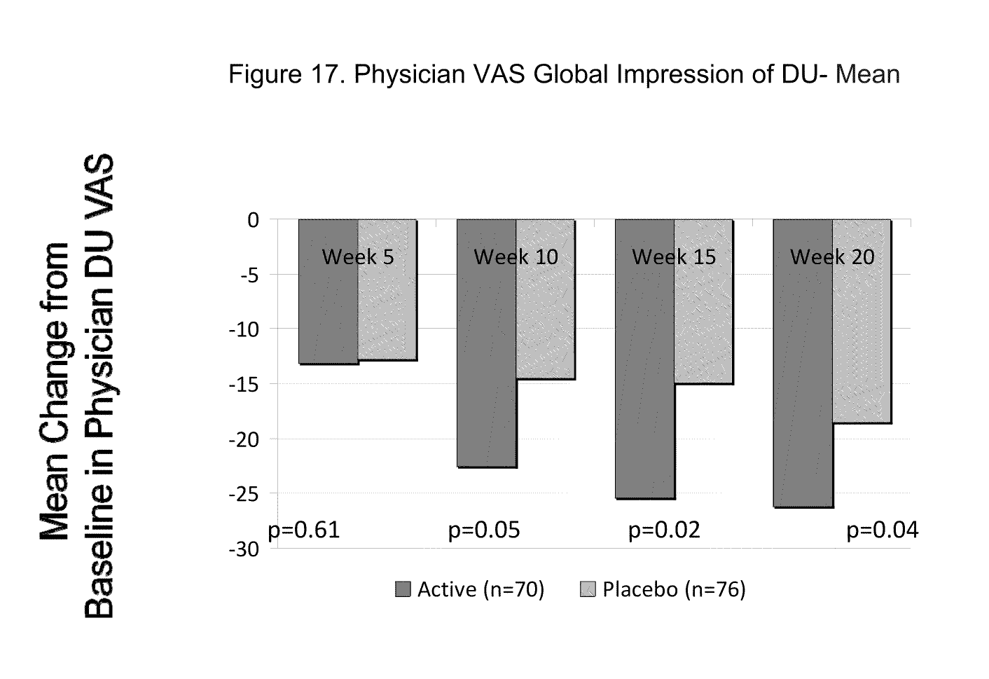

FIG. 17 presents physician VAS global impression of digital ulcers for total patient population.

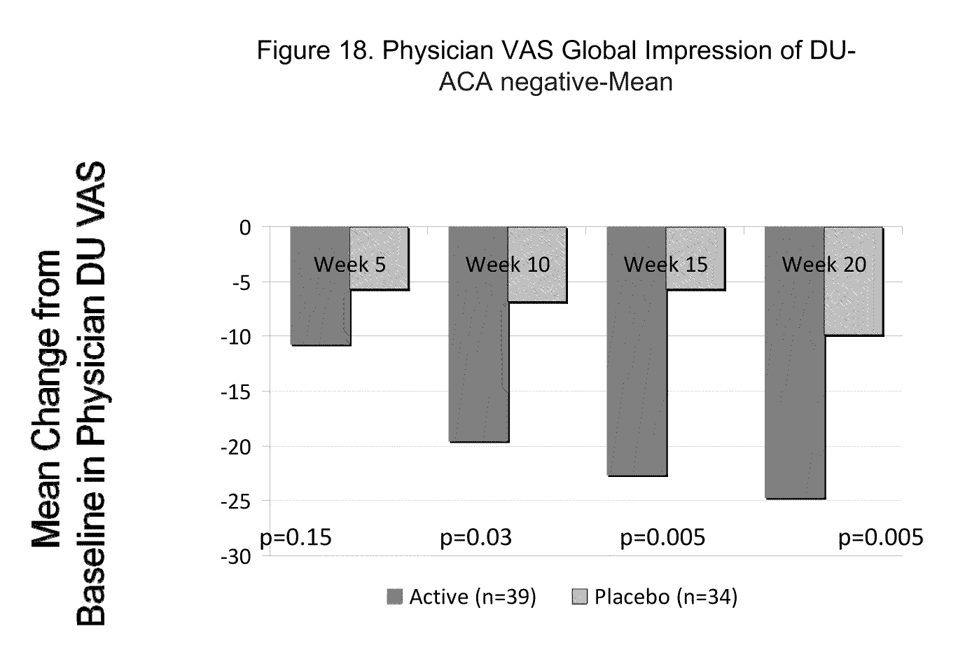

FIG. 18 presents physician VAS global impression of digital ulcers for ACA negative patient subgroup.

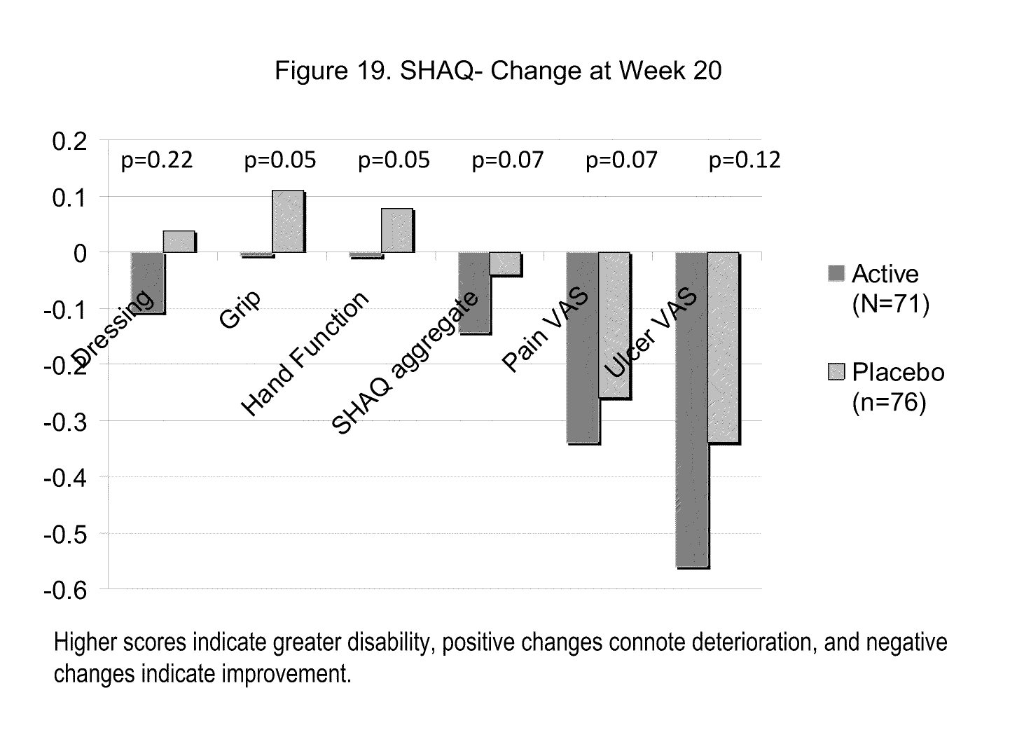

FIG. 19 presents SHAQ's change at Week 20.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention. All the publications, patent applications and patents cited in this specification are incorporated herein by reference in their entirety.

What is claimed is:

1. A method of treating a digital ischemic lesion and/or ameliorating or reducing at least one symptom or functional deficit associated with a digital ischemic lesion, comprising
orally administering to a subject in need thereof a formulation comprising an effective amount of treprostinil diethanolamine.

2. The method of claim 1, wherein the subject has a disease or condition selected from Raynaud's phenomenon, scleroderma, systemic sclerosis and a combination thereof and said digital ischemic lesion is caused by said disease or condition.

3. The method of claim 1, wherein the administering is performed twice daily.

4. The method of claim 1, wherein the administering results in at least one of a) increasing digital perfusion in the subject and b) increasing a digital skin temperature of the subject.

5. The method of claim 4, wherein the administering results in both a) increasing digital perfusion in the subject and b) increasing a digital skin temperature of the subject.

6. The method of claim 1, wherein said formulation is a sustained release oral formulation.

7. The method of claim 1, wherein said formulation is a tablet.

8. The method of claim 1, wherein the subject is a human being.

9. The method of claim 1, wherein the subject has a negative anti-centromere antibody status.

10. The method of claim 1, wherein said administering results in an increased hand function of the subject.

11. A method of treating a disease or condition selected from Raynaud's phenomenon, scleroderma, systemic sclerosis and a combination thereof, comprising
orally administering to a subject in need thereof a formulation comprising an effective amount of treprostinil diethanolamine.

12. The method of claim 11, wherein the administering is performed twice daily.

13. The method of claim 11, wherein the administering results in at least one of a) increasing digital perfusion in the subject and b) increasing a digital skin temperature of the subject.

14. The method of claim 13, wherein the administering results in both a) increasing digital perfusion in the subject and b) increasing a digital skin temperature of the subject.

15. The method of claim 11, wherein said formulation is a sustained release oral formulation.

16. The method of claim 11, wherein said formulation is a tablet.

17. The method of claim 11, wherein the subject is a human being.

18. The method of claim 11, wherein the subject has a negative anti-centromere antibody status.
